(19) United States
(12) Patent Application Publication
(10) Pub. No.: US 2026/0207399 A1
(43) Pub. Date: Jul. 23, 2026

(54) ABSORBENT PAD AND GARMENT COMPRISING THE ABSORBENT PAD

(71) Applicant: MAS Innovation (Private) Limited, Battaramulla (LK)

(72) Inventors: Mapitiyage Don Janith DUSHYANTHA, Battaramulla (LK); Kahandawala Arachchige Don Lakshan SANDARUWAN, Malabe (CA); Jithmi Mayondi DAYARTANE, Singapore (SG); Kalu Arachchige Manjula Nayanajith JAYAWARDANA, Malwana (LK); Uswaththa Arachchige Nimesh MADHURANGA, Malwana (LK)

(21) Appl. No.: 19/139,642

(22) PCT Filed: Dec. 15, 2023
(86) PCT No.: PCT/SG2023/050835

§ 371 Date: Jun. 16, 2025

(30) Foreign Application Priority Data
Dec. 16, 2022 (SG) 10202260483R
Dec. 16, 2022 (SG) 10202260484Q

(51) Int. Cl. A61F 13/538; A61F 13/15; A61F 13/42; A61F 13/53

(52) U.S. Cl. CPC A61F 13/538; A61F 13/15268; A61F 2013/15487; A61F 2013/422; A61F 2013/53016; A61F 2013/5383; A61F 2013/5386

(57) ABSTRACT

The present disclosure generally relates to an absorbent pad (20) for use in a 5 garment (30). The absorbent pad (20) A1 comprises a unitary absorbent layer (100) having a first surface (110) and an opposing second surface (120). The absorbent layer (100) is made of an absorbent fabric comprising a plurality of different yarns (200) knitted together between the first and second surfaces (110,120). The yarns (200) comprise first yarns (210) running in a wale direction for wicking fluid, second 10 yarns (220) running in a course direction for distributing fluid along the fabric (100), and third yarns (230) running in the course direction for absorbing fluid. The first yarns (210), second yarns (220), and third yarns (230) are interconnected to each other such that fluid is communicable between the first yarns (210), second yarns (220), and third yarns (230), the first surface (110) comprises substantially of the first 15 yarns (210), and the second surface (120) comprises substantially of the third yarns (230). The absorbent pad (20) further comprises a liquid impermeable barrier layer (400) attached to the absorbent layer (100) at the second surface (120) for retaining the absorbed fluid within the absorbent layer (100), wherein the first surface (110) is arranged to face towards a skin of a user when in use.

### CROSS REFERENCE TO RELATED APPLICATIONS

The present disclosure claims the benefit of Singapore Patent Application No. 10202260483R filed on 16 Dec. 2022 and Singapore Patent Application No. 10202260484Q filed on 16 Dec. 2022, each of which is incorporated in its entirety by reference herein.

### Technical Field

The present disclosure generally relates to an absorbent pad. More particularly, the present disclosure describes various embodiments of an absorbent pad having a unitary absorbent layer, as well as a garment comprising the absorbent pad.

### BACKGROUND

The listing or discussion of a prior-published document in this specification should not necessarily be taken as an acknowledgement that the document is part of the state of the art or is common general knowledge.

Absorbent pads and garments with such pads, such as reusable and washable absorbent undergarments, are worn with the purpose of absorbing bodily fluids. These bodily fluids include vaginal discharge, urine, menstrual fluid, sweat, and breast milk. Many people suffer from involuntary excretion of bodily fluids and there are few garments that have been designed to absorb such excretions that are functional and comfortable to the user wearing it.

For example, a woman who is menstruating will generally use a tampon or a sanitary pad, in addition to wearing an undergarment, to keep her outer garments from being soiled by menstrual fluid. While the tampon or pad often absorbs all the liquid flow, unexpected leaks can still occur. To avoid such leaks, she can instead choose to wear an adult brief, which offers a larger area of protection and may be particularly useful for women experiencing heavy menstrual flows. Adult briefs may also be useful for people who have urinary incontinence. However, adult briefs tend to be bulky and unattractive, making it difficult to conceal them under outer clothing, which may cause embarrassment to the user. Some disadvantages of wearing adult briefs and sanitary pads include prolonged exposure to wetness, which may result in discomfort, irritant dermatitis, and/or infections. Further, pads/tampons may be occasionally positioned incorrectly and adult briefs may be wrapped too loosely, both resulting in leakage. In addition, these conventional products are generally disposable, meaning that the environmental and economic costs can be significant.

Existing absorbent pads are usually composites of multiple layers, resulting in the fabrics being thick and uncomfortable, and the bulkiness of conventional products means that they do not allow the user to easily wear low-coverage undergarments. For example, absorbent pads must be placed into large, maximum-coverage undergarments that are capable of sufficiently containing them, while diapers and absorbent undergarments are generally large to adequately address the incontinence of the user. Therefore, someone who prefers thong-style or low-rise bikini underwear is often forced to choose between wearing preferred underwear, which risks leakage onto their outer clothing, or wearing a cumbersome and unattractive garment that would ensure that all leaks are prevented.

Therefore, in order to address or alleviate at least one of the aforementioned problems and/or disadvantages, there is a need to provide an improved absorbent pad and an improved garment comprising the absorbent pad.

### SUMMARY

According to a first aspect of the present disclosure, there is an absorbent pad for use in a garment. The absorbent pad comprises a unitary absorbent layer having a first surface and an opposing second surface. The absorbent layer is made of an absorbent fabric comprising a plurality of different yarns knitted together between the first and second surfaces. The plurality of different yarns comprise a plurality of first yarns running in a wale direction for wicking fluid, a plurality of second yarns running in a course direction for distributing fluid along the fabric, and a plurality of third yarns running in the course direction for absorbing fluid. The first yarns, second yarns, and third yarns are interconnected to each other such that fluid is communicable between the first yarns, second yarns, and third yarns, the first surface comprises substantially of the first yarns, and the second surface comprises substantially of the third yarns. The absorbent pad further comprises a liquid impermeable barrier layer attached to the absorbent layer at the second surface for retaining the absorbed fluid within the absorbent layer, wherein the first surface is arranged to face towards a skin of a user when in use.

According to a second aspect of the present disclosure, there is a garment comprising a fabric body, the absorbent pad according to the first aspect of the present disclosure, the pad being attached to the fabric body. The garment further comprises a liquid impermeable barrier layer being part of the fabric body and/or part of the absorbent pad.

An absorbent pad and a garment comprising the absorbent pad according to the present disclosure are thus disclosed herein. Various features, aspects, and advantages of the present disclosure will become more apparent from the following detailed description of the embodiments of the present disclosure, by way of non-limiting examples only, along with the accompanying drawings.

### BRIEF DESCRIPTION OF THE DRAWINGS

FIGS. 1A and 1B are illustrations of an absorbent pad for use in a garment and comprising a unitary absorbent layer, in accordance with embodiments of the present disclosure.

FIGS. 2A and 2B are illustrations of knitted yarns of the absorbent fabric.

FIGS. 3A to 3C are illustrations of the absorbent fabric with three different yarns.

FIGS. 4A to 4C are illustrations of the absorbent fabric with four different yarns.

FIGS. 5A and 5B are further illustrations of the absorbent fabric with different yarns.

FIG. 6 is an illustration of the absorbent pad comprising a top layer attached to the absorbent layer.

FIGS. 7A and 7B are illustrations of the absorbent pad being fastened to the garment.

FIGS. 8A and 8B are illustrations of the absorbent pad comprising an attaching element for attaching the absorbent pad to the garment.

FIGS. 9A to 9L are illustrations of the absorbent pad attached to the garment.

### DESCRIPTION

For purposes of brevity and clarity, descriptions of embodiments of the present disclosure are directed to an absorbent pad and a garment comprising the absorbent pad, in accordance with the drawings. While aspects of the present disclosure will be described in conjunction with the embodiments provided herein, it will be understood that they are not intended to limit the present disclosure to these embodiments. On the contrary, the present disclosure is intended to cover alternatives, modifications and equivalents to the embodiments described herein, which are included within the scope of the present disclosure as defined by the appended claims. Furthermore, in the following detailed description, specific details are set forth in order to provide a thorough understanding of the present disclosure. However, it will be recognized by an individual having ordinary skill in the art, i.e. a skilled person, that the present disclosure may be practiced without specific details, and/or with multiple details arising from combinations of aspects of particular embodiments. In a number of instances, known systems, methods, procedures, and components have not been described in detail so as to not unnecessarily obscure aspects of the embodiments of the present disclosure.

In embodiments of the present disclosure, depiction of a given element or consideration or use of a particular element number in a particular figure or a reference thereto in corresponding descriptive material can encompass the same, an equivalent, or an analogous element or element number identified in another figure or descriptive material associated therewith.

References to “an embodiment/example”, “another embodiment/example”, “some embodiments/examples”, “some other embodiments/examples”, and so on, indicate that the embodiment(s)/example(s) so described may include a particular feature, structure, characteristic, property, element, or limitation, but that not every embodiment/example necessarily includes that particular feature, structure, characteristic, property, element, or limitation. Furthermore, repeated use of the phrase “in an embodiment/example” or “in another embodiment/example” does not necessarily refer to the same embodiment/example.

The terms “comprising”, “including”, “having”, and the like do not exclude the presence of other features/elements/steps than those listed in an embodiment. Recitation of certain features/elements/steps in mutually different embodiments does not indicate that a combination of these features/elements/steps cannot be used in an embodiment. The terms “a” and “an” are defined as one or more than one. The use of “/” in a figure or associated text is understood to mean “and/or” unless otherwise indicated. The recitation of a particular numerical value or value range herein is understood to include or be a recitation of an approximate numerical value or value range.

The term “set” is defined as a non-empty finite organisation of elements that mathematically exhibits a cardinality of at least one (e.g. a set as defined herein can correspond to a unit, singlet, or single-element set, or a multiple-element set), in accordance with known mathematical definitions. The terms “first”, “second”, etc. are used merely as labels or identifiers and are not intended to impose numerical requirements on their associated terms.

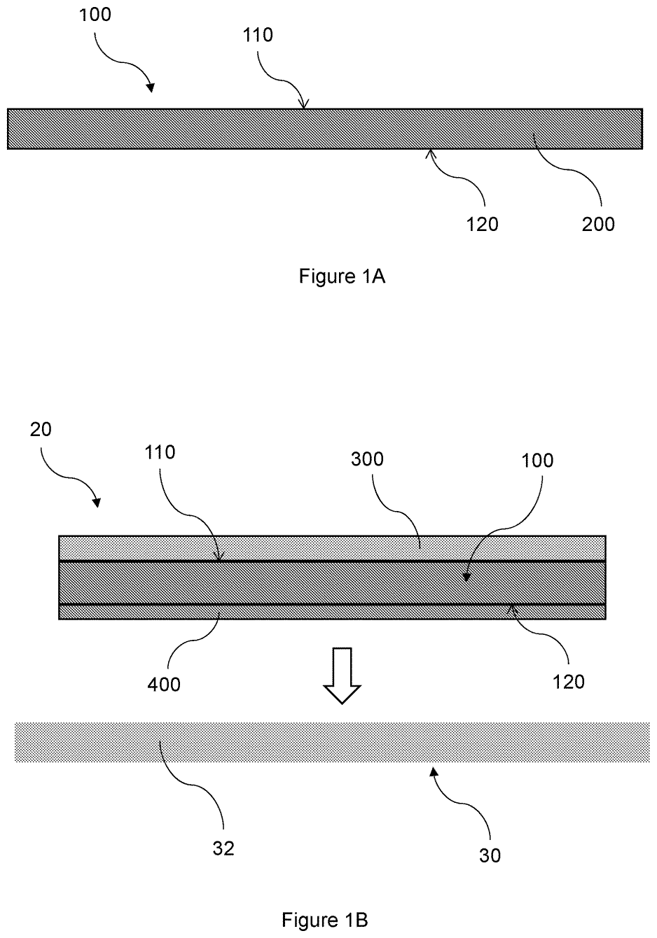

In representative or exemplary embodiments of the present disclosure, there is an absorbent pad 20, such as in but not limited to the form of a gusset, for use in a garment 30 as illustrated in FIGS. 1A and 1B. For example, the absorbent pad 20 is attachable to a fabric body 32 of the garment 30. For example, the absorbent pad 20 may be attached to the fabric body 32 via any means such as stitching, bonding, and/or fastening. For example, the absorbent pad 20 and the fabric body 32 may be attached by one or more of stitching (e.g. using a fusible yarn), ultrasonic welding, and an adhesive tape.

The garment 30 is worn by a user particularly at parts of the body where there may be excretions of bodily fluids, such as urine, menstrual blood, breast milk, saliva, and sweat. Examples of such garments 30 include brassieres, panties, lingerie, active wear, sportswear, swimwear, socks, footwear, and similar close-fitting or form-fitting garments.

The absorbent pad 20 includes a unitary absorbent layer 100 for absorbing liquid, such as the bodily fluids. The absorbent layer 100 has a first surface 110 and an opposing second surface 120. The first surface 110 is arranged to face towards a skin of the user when in use. The first surface 110 may be in contact with the user's skin when in use. In some embodiments as shown in FIG. 1B, the absorbent pad 20 may further include a top layer 300 attached to the absorbent layer 100 at the first surface 110, such that the top layer 300 faces towards the user's skin and may be in contact with the user's skin when in use. Further, the absorbent pad 20 includes a liquid impermeable barrier layer 400 attached to the absorbent layer 100 at the second surface 120 for retaining the absorbed fluid within the absorbent layer 100.

The absorbent layer 100 is made of an absorbent fabric comprising a plurality of different yarns 200 knitted together between the first surface 110 and second surface 120. Preferably, the yarns 200 are knitted together by warp knitting and in a single knitting run.

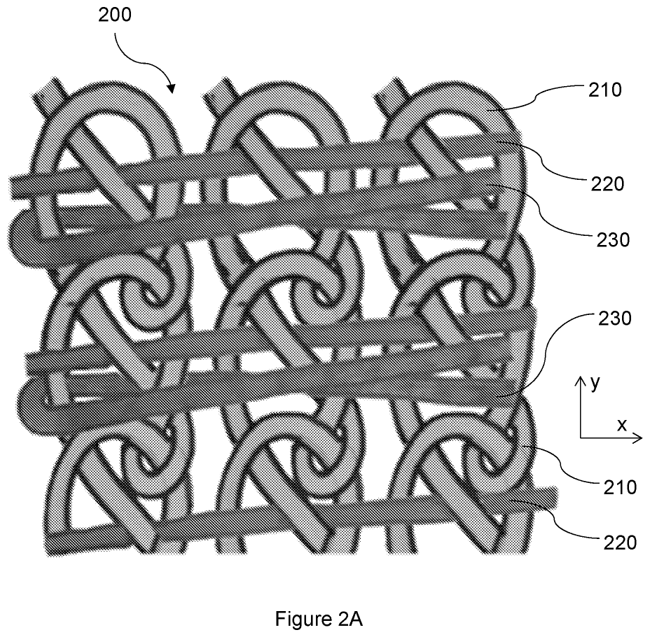

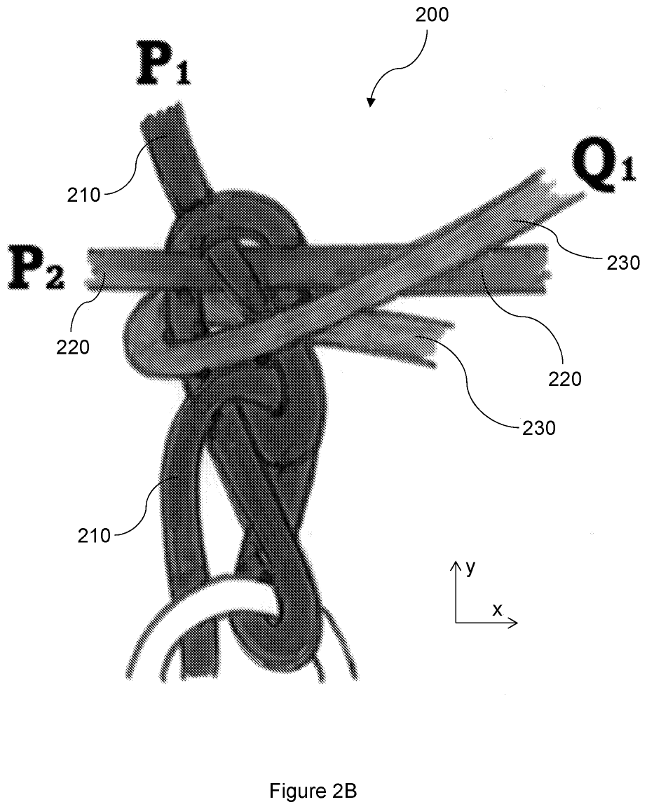

For example as shown in FIGS. 2A and 2B, the plurality of different yarns 200 include a plurality of first yarns 210, a plurality of second yarns 220, and a plurality of third yarns 230. The plurality of first yarns 210 run in a wale direction for wicking fluid. The plurality of second yarns 220 run in a course direction for distributing fluid along the absorbent layer 100. The plurality of third yarns 230 run in the course direction for absorbing fluid. Notably, wale refers to a column of loops running vertically (y-axis), and course refers to a crosswise row of loops running horizontally (x-axis).

The first yarns 210, second yarns 220, and third yarns 230 are interconnected to each other such that fluid is communicable between the first yarns 210, second yarns 220, and third yarns 230. Further, the first surface 110 comprises substantially of the first yarns 210. This may mean that the first yarns 210 form from 55% to 100%, such as 60% to 99%, 65% to 98%, 70% to 97%, 75% to 96%, 80% to 95%, or 85% to 90%, of the first surface 110. Further, the second surface 120 comprises substantially of the third yarns 230. This may mean that the third yarns 230 form from 55% to 100%, such as 60% to 99%, 65% to 98%, 70% to 97%, 75% to 96%, 80% to 95%, or 85% to 90%, of the second surface 120.

In some embodiments, the yarns 200 are knitted such that each of the first yarns 210 adopts a looping structure and each of the second yarns 220 is held in place by each loop of the first yarns 210 that each of the second yarns 220 contacts in the course direction. The first yarns 210 may be arranged in a half-tricot, tricot, single atlas, diamond net, or pillar stich arrangement.

In some embodiments, the yarns 200 are knitted such that each of the third yarns 230 is intermittently interlooped with the first yarns 210 such that at least some of the third yarns 230 extend outwardly from the second surface 120. The outward extension of the third yarns 230 create a lofted surface on the second surface 120. The third yarns 230 may be intermittently interlooped with the first yarns 210 at regular or irregular intervals. For example, the third yarns 230 are intermittently interlooped with the first yarns 210 from every second to every tenth loop of the first yarns 210 in the course direction.

In some embodiments, the first yarns 210 acquires fluid, for example when the first surface 110 contacts the user's skin, and wicks the fluid to the second yarns 220. The second yarns 220 distribute and spread the fluid along the absorbent layer 100, specifically along a plane parallel to the first surface 110 and second surface 120. The third yarns 230 receive the fluid from the second yarns 220 and absorb the fluid. The third yarns 230 are thus able to absorb fluid from the user, while the first surface 110, which comprises substantially of the first yarns 110, remains dry and comfortable for the user.

In some embodiments, the third yarns 230 are soaked with fluid, such as a liquid drug, and the second surface 120 is in contact with the user's skin. The third yarns 230 deliver the liquid drug through the second surface 120 to the user's skin, while the second yarns 220 and first yarns 210 prevent the liquid drug from leaking through the first surface 110.

In some embodiments, the third yarns 230 are soaked with fluid, such as a liquid drug, and the first surface 110 is in contact with the user's skin. The second yarns 220 receive the liquid drug from the third yarns 230 and spread the fluid along the absorbent layer 100. The first yarns 210 receive the liquid drug from the second yarns 220 and deliver the liquid drug through the first surface 110 to the user's skin. This allows for slow release of the liquid drug from the first surface 110, such as for skin care and cosmetic applications.

The first yarns 210 and second yarns 220 is made of a material suitable for wicking and/or distributing fluid, such as a hydrophobic material. This may be because the material is inherently hydrophobic in nature or because it has been treated to have hydrophobic properties (e.g. hydrophilic yarns such as wool or cotton that have been treated or functionalized with a hydrophobic treatment).

The material of the first yarns 210 and second yarns 220 may be naturally moisture-wicking and/or be treated to become moisture-wicking. For example, the material may be 100% polyester fabric with French Terry knit and a denier differential that assists in moving fluid. Other suitable materials include blends of polyester, polypropylene, polyethylene, nylon, regenerated materials (e.g. viscose blends), natural fibres (e.g. bamboo), and cotton. The material may optionally have triangular ridge structures of French Terry knitting that may face the user's skin, and the advantage of these structures is that less surface area comes into contact with the skin and therefore reduces any sensation of feeling wetness against the skin. The material may optionally have flat structures with a capillary gradient to generate differential capillary forces that move the liquid.

As an example, the material of the first yarns 210 and second yarns 220 may be 51% cotton. The material may be treated with a hydrophilic material or composition (e.g. polyethylene oxide, polyvinyl alcohol, polyacrylamide, poly acrylic acid, polyvinyl pyrrolidone, hydrophilic silicones, or hydrophilic polyurethanes) and/or a hydrophobic material or composition (e.g. silicones, polyfluoroalkyl acrylates, polyacrylates, polyurethanes, or waxes) to create a net hydrophilic gradient. This results in a combination of a “pushing” force generated by the hydrophobic properties and a “pulling” force generated by the hydrophilic properties that may wick any moisture or liquid through the first yarns 210 and second yarns 220.

The rate of wicking may be controlled to be faster or slower. The rate of wicking may be controlled by the density, thickness, or composition of the first yarns 210 and second yarns 220 and/or by the amount and type of hydrophobic and/or hydrophilic material applied to the first yarns 210 and second yarns 220. In another embodiment, the rate of wicking may be set such that the first surface 110 that may be in direct contact with the user feels dry or mostly dry to the user.

Additionally, the differential capillary forces can be created by the fabric structure of the absorbent layer 100 with different pore sizes that form funnel-like structures through the material in cross-section, where liquid is pulled from the larger pore sizes to the smaller pore sizes, due to the differential capillary pressure.

The third yarns 230 is made of an absorbent material suitable for absorbing and retaining fluid. The absorbent material may be inherently hydrophilic or may have been treated to provide a hydrophilic material. For example, a texturized polyester can be used to hold liquid, such as a polyester towel material. For example, the absorbent material may include cotton, a cotton blend, foam, a synthetic material, absorbent polymeric foam, a nanotechnology-based or-produced material, or any other moisture-absorbent material. The absorbent material may have a weight of 50 to 900 g/m², such as 180 to 300 g/m². For example, the absorbent material may be made from an 80:20 blend of polyester: nylon with a microfiber double terry knit. Other suitable absorbent materials include polypropylene, polyethylene, or any cellulose-based fabric and their blends including cotton, bamboo, etc.

In some embodiments, the absorbent material may be a 100% polyester double terry fabric. This material is approximately 90% air and so allows for a higher absorbent capacity, as moisture fills up the air gaps of the polyester terry fabric without significant expansion of the polyester fibres. This does not translate into significantly thicker absorbent layer 100. In some embodiments, the absorbent material may be made from a blended fibre comprising two or more of superabsorbent polymer (SAP), hydrogel, and polyester.

In some embodiments, the yarns 200 may include an antimicrobial agent or substance. For example, the antimicrobial substance may be one or more substances selected from the group consisting of a silver-containing substance, titanium dioxide, a quaternary silane, hydrogen peroxide, triclosan, and zinc pyrithione. Additionally or alternatively, the yarns 200 may include an anti-odour agent or substance that combats odour. For example, the substance that combats odour may be one or more substances selected from the group consisting of nanoparticles with acid-neutralising pockets, high surface area mineral compositions, high surface area ceramic compositions, high surface area clay compositions, and plant-based deodorizers such as essential mint oils. Further additionally or alternatively, the yarns 200 may include a stain-resistant, stain-release, or stain-proof agent or substance. Further additionally or alternatively, the yarns 200 may include a stretchable or elastic material to impart stretch and recovery properties to the absorbent layer 100, which can improve drapability, fit, and freedom of movement for the user.

Preferably, the absorbent pad 20 is washable and reusable while maintaining at least the initial absorbency of the absorbent layer 100. More preferably, the absorbent pad 20 is washable for at least 20 machine wash and dry cycles such that the absorbent fabric 100 remains reusable with at least 90% of the initial absorbency of the absorbent layer 100.

The absorbent layer 100 is formed as a single layer with different types of yarns 200 with different functionalities. Particularly, the absorbent layer 100 has first yarns 210 for fluid wicking, second yarns 220 for fluid distribution, and third yarns 230 for fluid absorption. These functionalities remain after multiple use, wash and dry cycles with insignificant functional and aesthetic change. Further, the various yarns 200 of the absorbent layer 100 are knitted in a single machine run such that the absorbent layer 100 is a unitary layer without any additional layers. This knitting of the yarns 200 allows the different yarns 200 to be simultaneously interconnected in a desirable manner to achieve the various yarn functionalities, including wicking, distribution, and absorption. This knitting process also allows the various yarn functionalities to be efficiently combined and integrated in the absorbent layer 100, such that the absorbent layer 100 has excellent fluid management and absorption properties, while minimizing the thickness of the absorbent layer 100.

The absorbent layer 100 may have various forms with various knitting configurations of the different yarns 200, including but not limited to the first yarns 210, second yarns 220, and third yarns 230. It will be appreciated that the distribution components (second yarns 220) interconnect the acquisition components (first yarns 210) and the absorbent components (third yarns 230). The distribution components receive the fluid acquired by the acquisition components and distributes along the absorbent layer 100 for evenly distributed retention within the absorbent components. The knitting and interloping interconnection between the various yarns 200 enable the distribution components to be connected with the acquisition components and the absorbent components, hence the distribution components can distribute fluid across the acquisition components and the absorbent components as well to facilitate wicking. The absorbent components absorb and retain the liquid transported by the distribution components. The absorbent components are interconnected closely with the distribution components, and the high liquid retention capability of the material of the absorbent components ensures high liquid retention capacity of the absorbent layer 100.

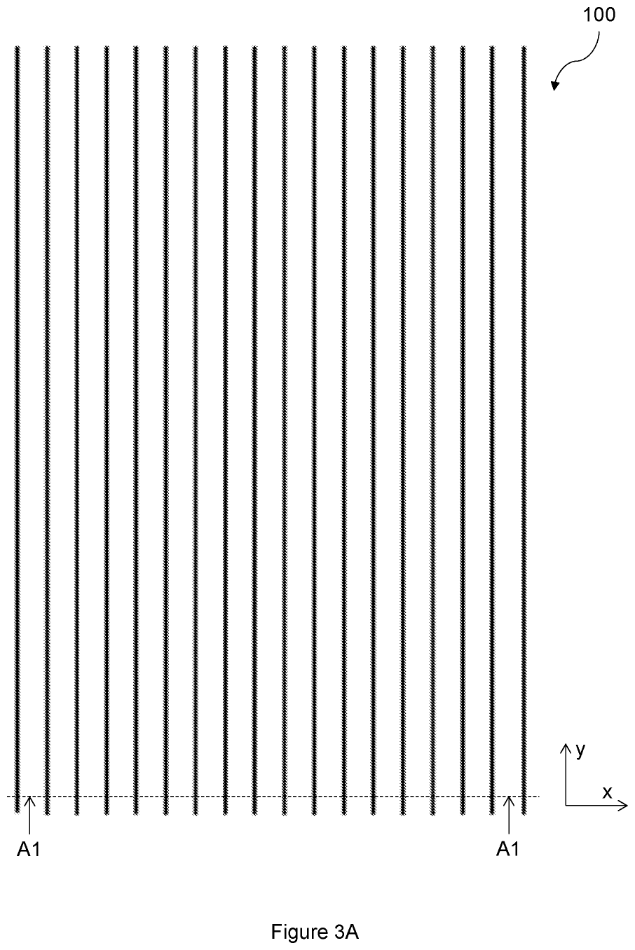

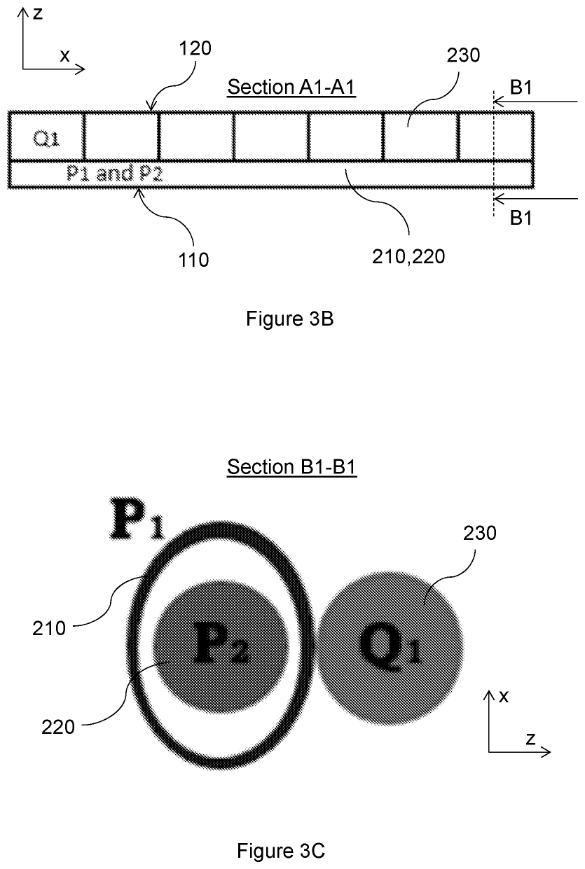

In some embodiments as shown in FIGS. 3A to 3C, the different yarns 200 of the absorbent layer 100 include the first yarns 210 running in the wale direction for wicking fluid, the second yarns 220 running in the course direction for distributing fluid along the absorbent layer 100, and the third yarns 230 running in the course direction for absorbing fluid. The first yarns 210, second yarns 220, and third yarns 230 are designated as P1, P2, and Q1, respectively. Notably, the first surface 110 comprises substantially of the first yarns 210 (P1), and the second surface 120 comprises substantially of the third yarns 230 (Q1).

Each of the first yarns 210 (P1) may adopt a looping structure and each of the second yarns 220 (P2) may be held in place by each loop of the first yarns 210 (P1) that each of the second yarns 220 (P2) contacts in the course direction. Each of the third yarns 230 (Q1) may be intermittently interlooped with the first yarns 210 (P1) such that at least some of the third yarns 230 (Q1) extend outwardly from the second surface 120, thereby forming a lofted second surface 120. The third yarns 230 may be intermittently interlooped with the first yarns 210 at regular or irregular intervals, such as from every second to every tenth loop of the first yarns 210 in the course direction.

The first yarns 210 (P1) and second yarns 220 (P2) may include a suitable wicking material as mentioned above. For example, the material for the first yarns 210 (P1) and second yarns 220 (P 2) may have a Denier value of 30 D to 1000 D. For example, the material for the first yarns 210 (P1) may have a Denier value of 30 D to 50 D. For example, the material for the second yarns 220 (P2) may have a Denier value of 100 D to 150 D. The third yarns 230 (Q1) may include a suitable absorbent material as mentioned above. For example, the material for the third yarns 230 (Q1) may have a Denier value of 30 D to 2000 D. For example, the material for the third yarns 230 (Q1) may have a Denier value of 800 D to 1200 D.

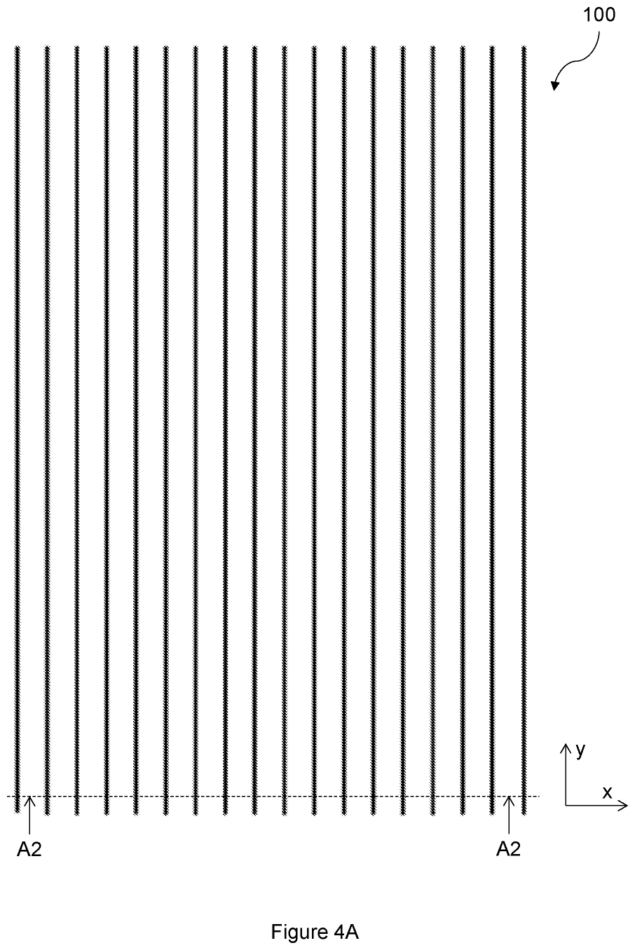

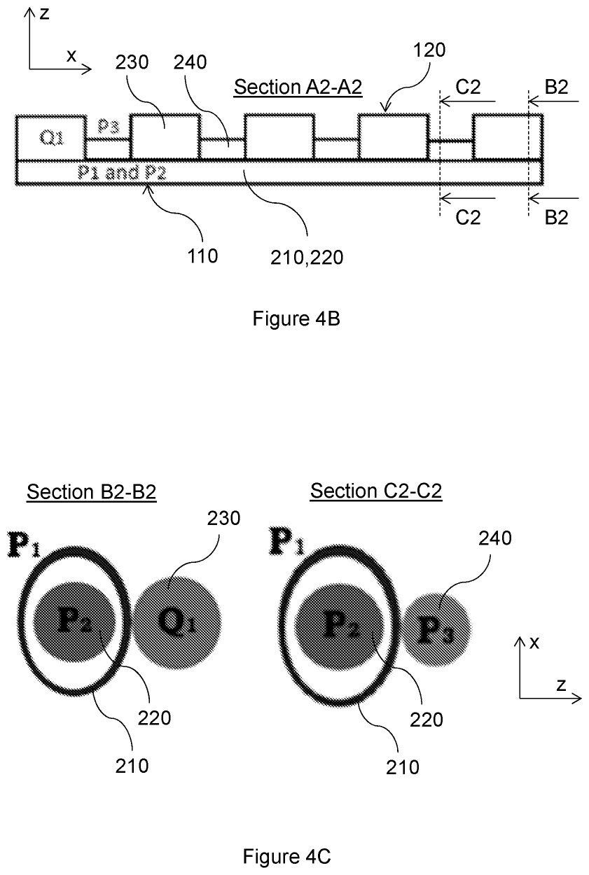

In some embodiments as shown in FIGS. 4A to 4C, the different yarns 200 of the absorbent layer 100 include the first yarns 210 running in the wale direction for wicking fluid, the second yarns 220 running in the course direction for distributing fluid along the absorbent layer 100, and the third yarns 230 running in the course direction for absorbing fluid. The different yarns 200 further include a plurality of fourth yarns 240 running in the course direction for distributing fluid along the absorbent layer 100, similar to the functionality of the second yarns 220. The first yarns 210, second yarns 220, third yarns 230, and fourth yarns 240 are designated as P1, P2, Q1, and P3, respectively. The first surface 110 may comprise substantially of the first yarns 210 (P1), and the second surface 120 may comprise substantially of the third yarns 230 (Q1) and fourth yarns 240 (P3). Alternatively, the first surface 110 may comprise substantially of the first yarns 210 (P1) and fourth yarns 240 (P3), and the second surface 120 may comprise substantially of the third yarns 230 (Q1).

Each of the first yarns 210 (P1) may adopt a looping structure and each of the second yarns 220 (P2) may be held in place by each loop of the first yarns 210 (P1) that each of the second yarns 220 (P2) contacts in the course direction. Each of the third yarns 230 (Q1) may be intermittently interlooped with the first yarns 210 (P1) such that at least some of the third yarns 230 (Q1) extend outwardly from the second surface 120, thereby forming a lofted second surface 120. The third yarns 230 may be intermittently interlooped with the first yarns 210 at regular or irregular intervals, such as from every second to every tenth loop of the first yarns 210 in the course direction. The fourth yarns 240 (P3) may be intermittently interlooped with the first yarns 210 (P1), such that the fourth yarns 240 (P3) intermittently separate the third yarns 230 (Q1).

The third yarns 230 and fourth yarns 240 may have an alternating arrangement along each course, such as shown in FIG. 4B. Further, the third yarns 230 (Q1) may be thicker than the fourth yarns 240 (P3). This may result in portions of the second surface 120 comprising the third yarns 230 (Q1) being thicker than portions of the second surface 120 comprising the fourth yarns 240 (P3), such as shown in FIG. 4B. Alternatively, the fourth yarns 240 (P3) may be thicker than the third yarns 230 (Q1), which may result in portions of the second surface 120 comprising the fourth yarns 240 (P3) being thicker than portions of the second surface 120 comprising the third yarns 230 (Q1). Yet alternatively, the third yarns 230 (Q1) and fourth yarns 240 (P3) have substantially equal thickness, which may result in the second surface 120 comprising substantially of the third yarns 230 (Q1) and fourth yarns 240 (P3) having substantially equal thickness. It will be appreciated that the thickness of the yarns 200 may be adapted and used for aesthetic purposes, such as for sight and/or feel. For example, the thickness of the third yarns 230 (Q1) and fourth yarns 240 (P3) may be varied within a course, or within courses to create a desired texture.

The first yarns 210 (P1), second yarns 220 (P2), and fourth yarns 240 (P3) may include a suitable wicking material as mentioned above. For example, the material for the first yarns 210 (P1), second yarns 220 (P2), and fourth yarns 240 (P3) may have a Denier value of 30 D to 1000 D. For example, the material for the first yarns 210 (P1) may have a Denier value of 30 D to 50 D. For example, the material for the second yarns 220 (P2) may have a Denier value of 100 D to 150 D. For example, the material for the fourth yarns 240 (P3) may have a Denier value of 30 D to 1000 D. The third yarns 230 (Q1) may include a suitable absorbent material as mentioned above. For example, the material for the third yarns 230 (Q1) may have a Denier value of 30 D to 2000 D. For example, the material for the third yarns 230 (Q1) may have a Denier value of 800 D to 1200 D.

In some embodiments as shown in FIG. 4B, the third yarns 230 (Q1) and fourth yarns 240 (P3) occupy the same surface (second surface 120) of the absorbent layer 100. In other words, the second surface 120 comprises substantially of the third yarns 230 (Q1) and fourth yarns 240 (P3). In some other embodiments, the fourth yarns 240 (P3) may occupy the opposing surface (first surface 110) instead. In other words, the first surface 110 comprises substantially of the first yarns 210 (P1) and fourth yarns 240 (P3). For example, each of the first yarns 210 (P1) may adopt a looping structure and each of the fourth yarns 240 (P3) may be held in place by each loop of the first yarns 210 (P1) that each of the fourth yarns 240 (P3) contacts in the course direction. Further, the third yarns 230 (Q1) and fourth yarns 240 (P3) may be present across the entire course on the respective opposing surfaces. This may provide additional wicking properties that may be useful in certain applications.

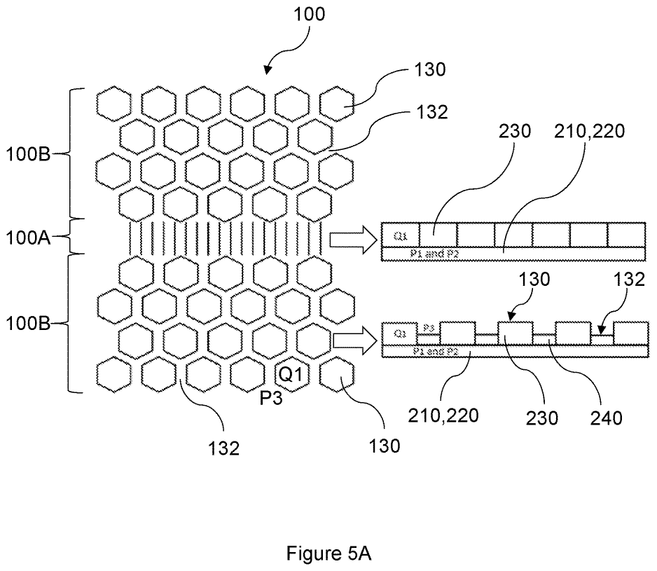

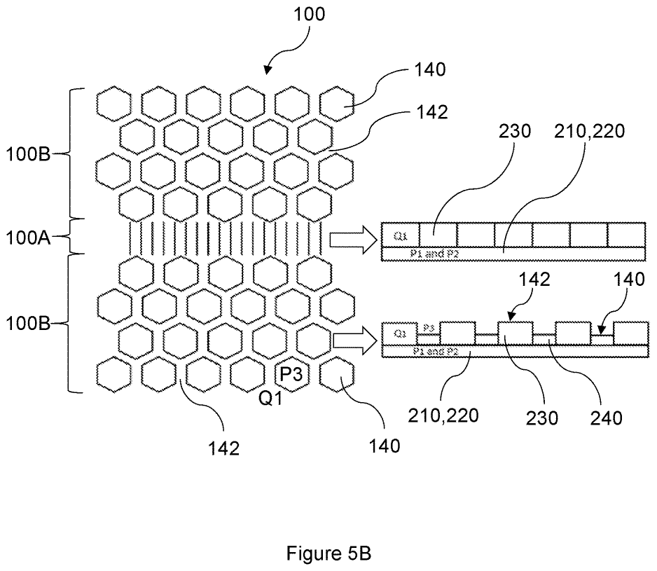

In some embodiments as shown in FIGS. 5A and 5B, the absorbent layer 100 may be divided into a plurality of different regions. For example, the absorbent layer 100 may be divided into one or more first regions 100A and one or more second regions 100B. Alternatively, the absorbent layer 100 may comprise only the first regions 100A or only the second regions 100B.

In one embodiment as shown in FIG. 5A, the first region 100A includes the first yarns 210 (P1), second yarns 220 (P2), and third yarns 230 (Q1), similar to the embodiments shown in FIGS. 3A to 3C. The second region 100B includes a set of absorbent islands 130 and a set of wicking seas 132 separating the absorbent islands 130 from each other. Each absorbent island 130 includes the first yarns 210 (P1), second yarns 220 (P2), and third yarns 230 (Q1), and each wicking sea 132 includes the first yarns 210 (P1), second yarns 220 (P2), and fourth yarns 240 (P3), similar to the embodiments shown in FIGS. 4A to 4C. Each absorbent island 130 may be defined in a hexagonal shape. It will be appreciated that the absorbent island 130 may be defined in any shape, and that each wicking sea 132 may similarly be defined in any shape.

In one embodiment as shown in FIG. 5B, the first region 100A includes the first yarns 210 (P1), second yarns 220 (P2), and third yarns 230 (Q1), similar to the embodiments shown in FIGS. 3A to 3C. The second region 100B includes a set of wicking islands 140 and a set of absorbent seas 142 separating the wicking islands 140 from each other. Each wicking island 140 includes the first yarns 210 (P1), second yarns 220 (P2), and fourth yarns 240 (P3), and each absorbent sea 142 includes the first yarns 210 (P1), second yarns 220 (P2), and third yarns 230 (Q1), similar to the embodiments shown in FIGS. 4A to 4C. Each wicking island 140 may be defined in a hexagonal shape. It will be appreciated that the wicking island 140 may be defined in any shape, and that each absorbent sea 142 may similarly be defined in any shape.

As shown in FIG. 5A, the third yarns 230 (Q1) and fourth yarns 240 (P3) in the second region 100B alternate along each course to form the absorbent islands 130 and wicking seas 132. As shown in FIG. 5B, the third yarns 230 (Q1) and fourth yarns 240 (P3) in the second region 100B alternate along each course to form the wicking islands 140 and absorbent seas 142. The second region 100B thus has an alternating arrangement of the third yarns 230 (Q1) and fourth yarns 240 (P3). It will be appreciated that the yarns 200 may be arranged in various ways and is not limited to the shapes and patterns shown in FIGS. 5A and 5B, such as the hexagonal absorbent islands 130 and hexagonal wicking islands 140. It will be appreciated that the thickness of the yarns 200, including the first yarns 210 (P1), second yarns 220 (P2), third yarns 230 (Q1), and/or fourth yarns 240 (P3), may be adapted and used for aesthetic purposes, such as for sight and/or feel.

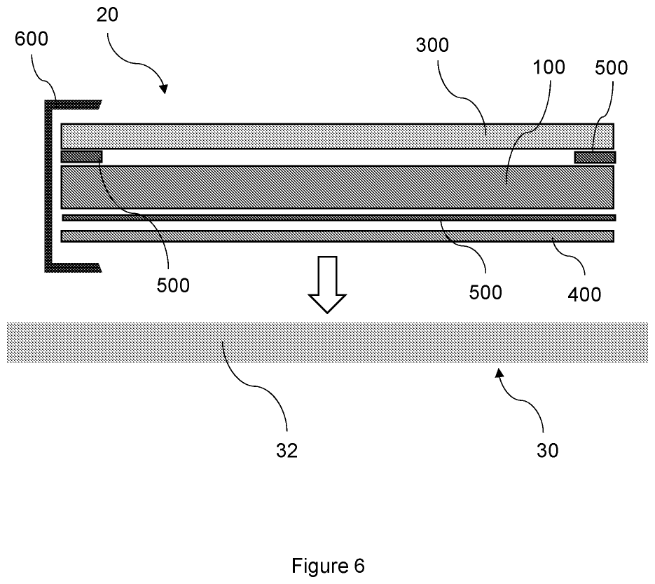

As described above as shown in FIGS. 1B and 6, the absorbent pad 20 further includes a liquid impermeable barrier layer 400 attached to the absorbent layer 100 at the second surface 120 for retaining the absorbed fluid within the absorbent layer 100. The barrier layer 400 is leak-proof and may include any known wholly or partially liquid-blocking material. Preferably, the barrier layer 400 is breathable, so that liquid may not pass through it, but gases (including water vapour) can do so. For example, the barrier layer 400 may include one or more thermoplastic films and/or one or more thermoset films, where the thermoplastic or thermoset film is selected from one or more of the group consisting of polyurethane, polyester, polyolefin, and silicone. Particular examples of liquid impermeable materials include layers made from a liquid impermeable polymer or a thermoplastic polyurethane film. The barrier layer 400 may alternatively or additionally include or be made of liquid impermeable fabrics and/or fusible yarns.

In some embodiments, the barrier layer 400 may be a lightweight tightly knitted/woven fabric coated with hydrogel or treated with hydrophobic finishes such as durable water repellents (e.g. DWR), or the barrier layer 400 may be a lightweight tightly knitted/woven fabric made using textile/SAP hybrid fibres. Alternatively, the barrier layer 400 may be a liquid-proof membrane present independently or laminated/attached to a textile material with adhesive bonding or other attachment methods including ultrasonic bonding or stitching.

When used in a garment 30 comprising the absorbent pad 20, the barrier layer 400 may provide the advantage of being fully breathable in dry form, while providing an effective barrier material upon exposure to liquid. Furthermore, these materials may also enable the absorbent pad 20 to dry more quickly than the use of a liquid impermeable polymer such as a thermoplastic polyurethane film.

In some embodiments, the barrier layer 400 is a separate layer that is attached to the second surface 120 of the absorbent layer 100. In some embodiments, the barrier layer 400 is part of the absorbent layer 100. For example, the second surface 120 of the absorbent layer 100 may be treated or laminated to provide, or incorporated with, a liquid impermeable barrier. For example, a portion of the thickness of the absorbent layer 100 from the second surface 120 may function as the liquid impermeable barrier layer 400. In some embodiments, the barrier layer 400 is part of the fabric body 32 of the garment 30 to which the absorbent pad 20 is attached. For example, the fabric body 32 may be made of or include a hydrophobic material or treated with a hydrophobic or waterproofing agent to achieve the liquid impermeable property. This may help to reduce the overall size and thickness of the absorbent pad 20.

Some suitable treatments applicable to the fabric body 32 and absorbent layer 100 to form the barrier layer 400 may include any suitable material that results in liquid impermeable properties. Examples of such materials include, but are not limited to, hydrophobic-and water-repellent finishes. Such finishes may include, but are not limited to wax-based repellents, silicone-base repellents and non-fluorocarbon-based repellents. Alternatively or additionally, the fabric body 32 or the second surface 120 of the absorbent layer 100 may be coated with a suitable material, which may include, but is not limited to an SAP/hydrogel coating or a thermoplastic and/or thermoset coating, wherein the latter two coatings may be derived from polymers selected from one or more of the group of polyurethane, polyester, poly-olefin and silicone polymers.

Preferably, the barrier layer 400 is a separate layer that is attached to the absorbent layer 100. For example, the barrier layer 400 may be attached by lamination across the second surface 120. Alternatively, the barrier layer 400 may be attached at a plurality of discrete points (e.g. adhesive points or stitched/knitted points) across at least part of the second surface 120. In embodiments where the top layer 300 is present in the absorbent pad 20, the top layer 300 may similarly be attached to the absorbent layer 100 by lamination or at a plurality of discrete points (e.g. adhesive points or stitched/knitted points).

As used herein, the term “adhesive” or “adhesive material” is a material that enables objects to be adhered to each other. For example, the adhesive material may include a material that becomes liquid impermeable after curing, such as a thermoplastic or thermoset glue. For example, the adhesive material may be in the form of liquid glue, such as hot-melt glue, drop-on-demand glue, line-on-demand glue, liquid resin, or the like, or combinations thereof. For example, the adhesive material may be in the form of an adhesive film or tape, such as a single-sided or double-sided adhesive film or tape.

The absorbent pad 20 may include adhesive films 500 for bonding various layers, components, and/or parts of the absorbent pad 20. For example as shown in FIG. 6, peripheries of the absorbent layer 100 and top layer 300 are attached together by adhesive films 500. For example, peripheries of the absorbent layer 100 and barrier layer 400 by an adhesive film 500. The adhesive films 102 may be applied across at least part of, or the entire, respective surfaces between the absorbent layer 100, top layer 300, and barrier layer 400.

The adhesive film 500 may include an adhesive tape, liquid glue, or hotmelt powder glue). When the adhesive film 500 is an adhesive tape, the tape may be a double-sided adhesive tape and said tape may have a single layer or multiple layers where said multiple layers may have one or more functions, such as liquid barrier functions, elastic functions, etc. For example, the adhesive tape is a Bemis tape. When the adhesive is a liquid glue, the glue may be a hot melt glue, a liquid resin or combinations thereof (e.g. the adhesive may be a hot melt glue/liquid resin bonding by nozzle extrusion or liquid resin bonding by screen printing/template printing). Alternatively, the adhesive film 500 may be replaced by an adhesive or bonding process such as ultrasonic bonding. In some cases, the adhesive film 500 may be replaced by stitching.

In some embodiments as shown in FIG. 6, the absorbent pad 20 includes a liquid impermeable sealing element 600 attached to peripheries of the absorbent layer 100 and liquid impermeable barrier layer 400. The sealing element 600 may include a single-sided adhesive tape and said tape may have a single layer or multiple layers where said multiple layers may have one or more functions, such as liquid barrier functions, elastic functions, etc. The sealing element 600 preferably has a C-fold or C-shaped arrangement that entirely encloses the peripheries of the absorbent layer 100 and barrier layer 400. This arrangement forms a liquid impermeable barrier seal or cuff and advantageously reduces leakage of bodily fluids from the absorbent pad 20 onto the garment 30. This arrangement also results in a durable garment 30 that may be washed and reused multiple times.

In some embodiments, the top layer 300 may function as a wicking layer to facilitate liquid transfer to the absorbent layer 100. This may be accomplished through the use of specific materials, such as a 100% polyester French terry fabric or a 100% polyester double terry fabric. Suitable materials for the wicking layer include polyamide, polyester, polyolefin, polyurethane, polyacrylonitrile, patural cellulose, regenerated cellulose, regenerated cellulose derivatives (i.e. cellulose acetate and cellulose triacetates), natural protein and regenerated protein and any other wicking material known in the art. For example, the wicking layer may be made from fibres or yarns made with fibres, where said fibres and yarns are selected from one or more of the group consisting of polyamide, polyester, polyolefin, polyurethane, polyacrylonitrile, natural cellulose, regenerated cellulose, regenerated cellulose derivatives (i.e. cellulose acetate and cellulose triacetates), natural protein and regenerated protein. The wicking layer may be produced using technologies such as knitting (warp knitting such as raschel Tricot, weft knitting such as circular or flat), weaving, non-woven methods (blow spinning, staple nonwoven, spun laid, air-laid, needle punched, thermal bonded, hydro-entangled, chemical bonded and so forth), electro-spinning, force-spinning etc. The wicking layer may also include one or more of the coatings, treatments encapsulation or entrapments, which would enhance its liquid and moisture management functionality, such as rate of absorbency/wicking, absorption capacity, rate of spreading and distribution, one way liquid transport etc. The wicking layer may include an antimicrobial agent, anti-odour agent, stain-resistant agent, and/or elastic material. It will be appreciated that materials and structures described above for the yarns 200 may apply similarly or analogously to the wicking layer, and vice versa.

In some embodiments, the top layer 300 may include one or more of a hydrophilic material, a hydrophobic material, and a hydrochromic material. For example, the top layer 300 includes the hydrochromic material that is configured to change colour in response to contact with liquid. The hydrochromic material may allow one to introduce various colours and designs to the aesthetic appearance of the absorbent pad 20. For example, the hydrochromic material includes hydrochromic prints and water-based pigments. An example of a hydrochromic print is a printing paste comprising an ink that is normally opaque coloured but will become transparent when wetted. Such printing ink can be used beneath a white-coloured coating to incorporate hidden effects that become exposed when wetted. The printed area would return to its original opaque coloured appearance when dried.

The garment 30 may include one or more elastic bands 34 to provide flexibility/elasticity/stretchability to the user. For example, the elastic bands 34 may be lined on or attached to the fabric body 32, such as by adhesive or ultrasonic bonding. The elastic bands 34 allow the garment 30 to come in a small number of discrete sizes that are able to cater to a wide variety of users of various sizes. For example, the elastic bands 34 are arranged to surround the waist opening and/or leg openings of the garment 30.

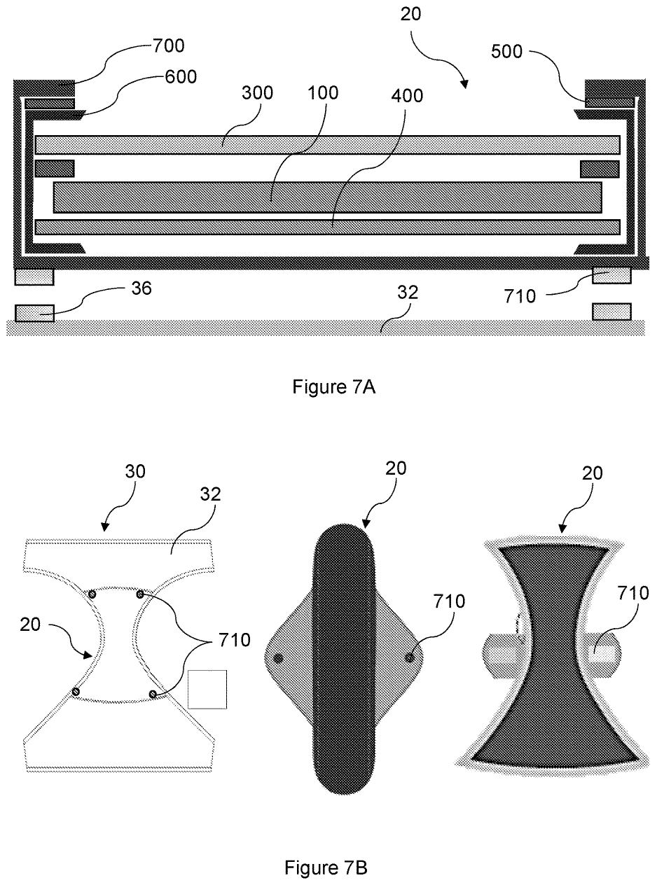

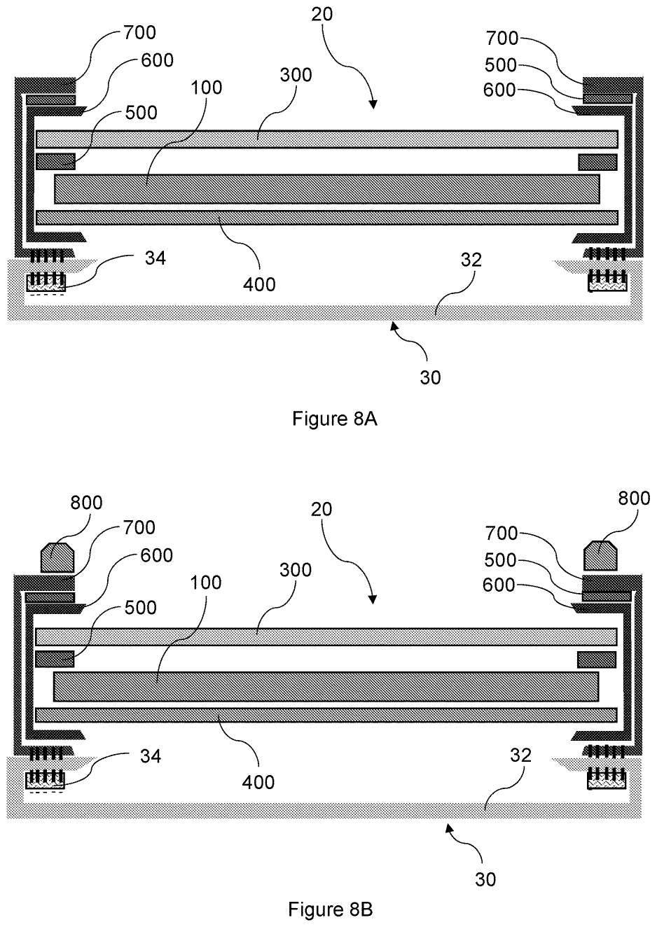

In some embodiments as shown in FIGS. 7A, 8A, and 8B, the absorbent pad 20 may include an attaching element 700 for attaching the absorbent pad 20 to the fabric body 32, such as by stitching and/or bonding. The attaching element 700 may have a C-fold or C-shaped arrangement, like that of the sealing element 600, that entirely covers the sealing element 600. The attaching element 700 may be formed of a suitable material that is preferably liquid impermeable. For example, the attaching element 700 may be formed of the same, similar, or different fabric or textile material as that of the fabric body 32. For example, the attaching element 700 comprises a liquid impermeable or hydrophobic material such as a durable water repellent or a silicone material. The attaching element 700 may be treated with a hydrophobic or waterproofing agent, such as a durable water repellent that forms a coating on the attaching element 700.

As shown in FIGS. 7A and 7B, the absorbent pad 20 may include a plurality of fasteners 710 attached to the attaching element 700. The fasteners 710 are configured for fastening the absorbent pad 20 to the garment 30. More specifically, the fasteners 710 are configured for engaging with corresponding fasteners 36 of the garment 30, wherein the fasteners 36 may be located on the inner side and/or outer side of the fabric body 32. The fasteners 36, 710 may include snap buttons and/or touch fasteners.

As shown in FIGS. 8A and 8B, the garment 30 may include one or more elastic bands 34 to provide flexibility/elasticity/stretchability to the user. For example, the elastic bands 34 may be lined on or attached to the fabric body 32, such as by adhesive or ultrasonic bonding. The elastic bands 34 allow the garment 30 to come in a small number of discrete sizes that are able to cater to a wide variety of users of various sizes. For example, the elastic bands 34 are arranged to surround the waist opening and/or leg openings of the garment 30.

As shown in FIG. 8B, the attaching element 700 may include a silicone lining or extrusion thereon. The attaching element 700 may form a liquid impermeable ridge 800 on the periphery of the absorbent pad 20. This liquid impermeable ridge 800 facilitates channelling of liquid to the absorbent pad 20 and reduces overflow from the peripheries of the absorbent pad 20.

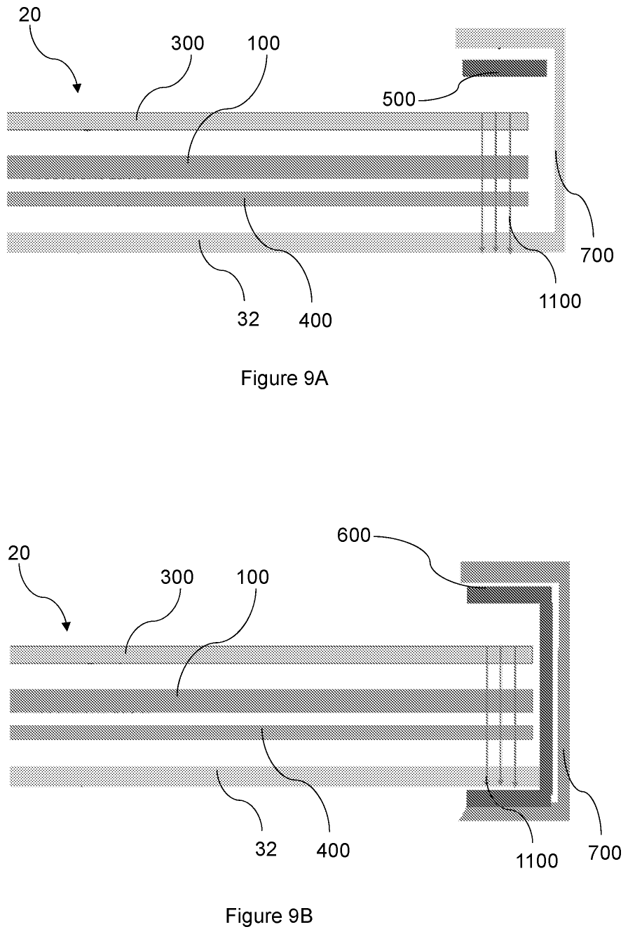

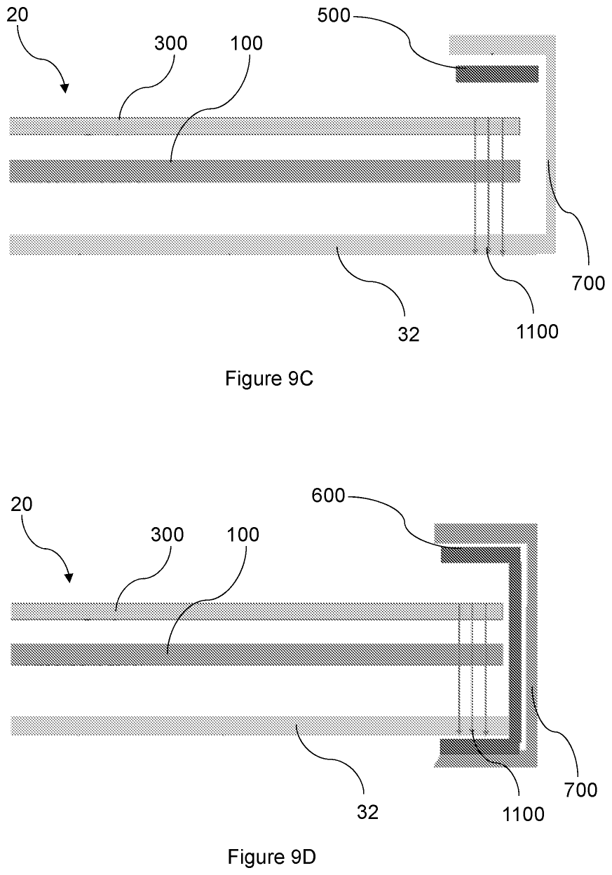

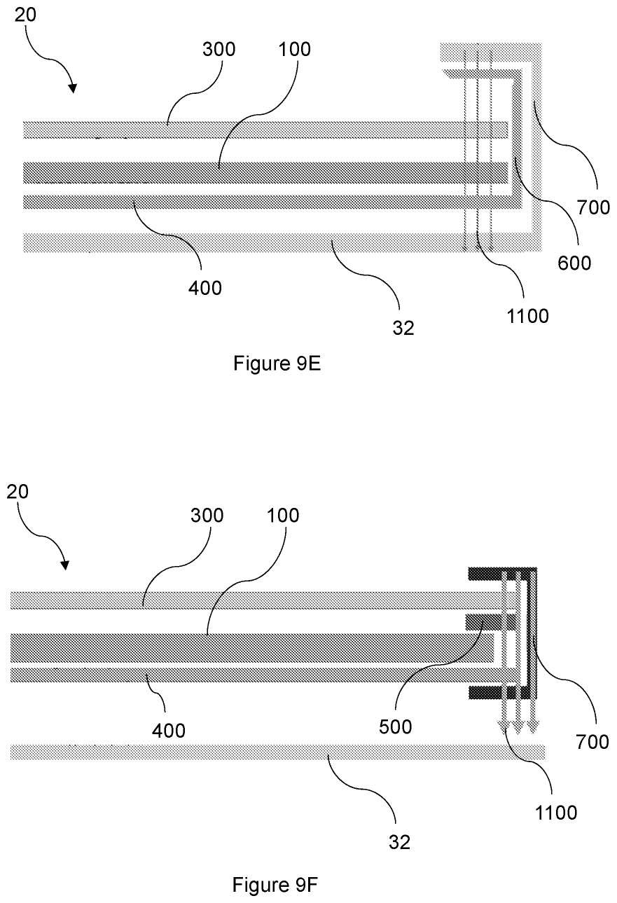

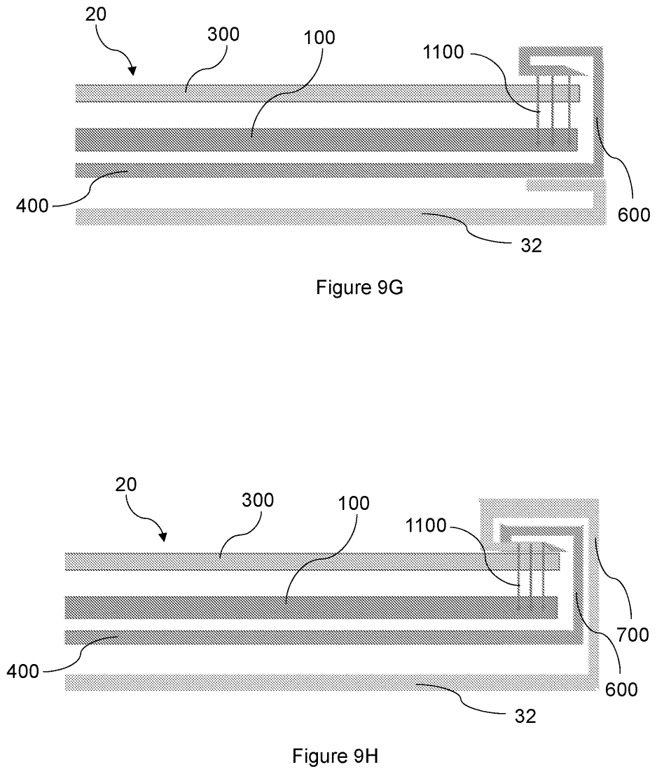

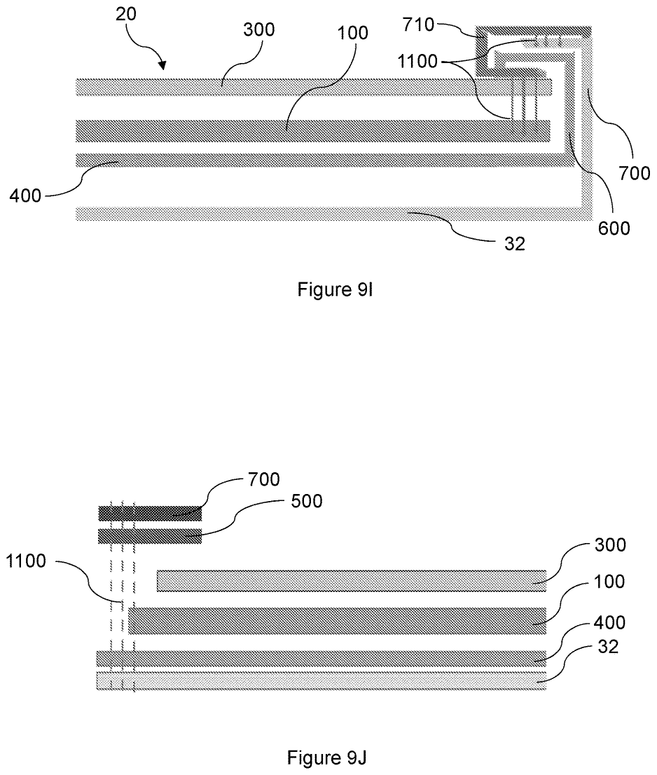

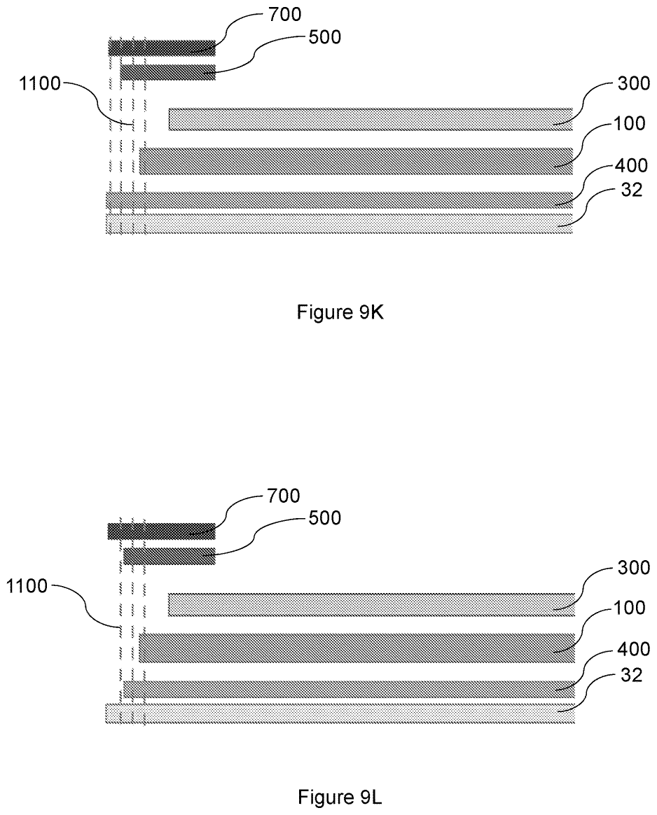

FIGS. 9A to 9L show various ways of attaching the absorbent pad 20 to the garment 30, specifically by attaching the attaching element 700 to the fabric body 32. Although these embodiments show the absorbent pad 20 comprising the top layer 300 attached to the absorbent layer 100, it will be appreciated that the absorbent pad 20 may not have the top layer 300, since the absorbent layer 100 is a unitary layer capable of both wicking and absorbing fluid.

In one embodiment as shown in FIG. 9A, the absorbent pad 20 is stitched to the fabric body 210. The attaching element 700 is an extension of the fabric body 32 and is attached to the top layer 300 via an adhesive film 500. The fabric body 32 acts as a liquid impermeable barrier and the attaching element 700 is similarly liquid impermeable. The attaching element 700 and fabric body 32 cooperatively cover the visible stitch lines 900 and seal the periphery of the absorbent pad 20 from side leakages.

In one embodiment as shown in FIG. 9B, the absorbent pad 20 is stitched to the fabric body 32. The sealing element 600 is attached to the top layer 300 and fabric body 32. Additionally, the attaching element 700 is attached to the sealing element 600 to cover the sealing element 600. The sealing element 600 and attaching element 700 cooperatively cover the visible stitch lines 900 and seal the periphery of the absorbent pad 20 from side leakages.

In one embodiment as shown in FIG. 9C, the absorbent pad 20 includes the absorbent layer 100 and top layer 300, but does not have the barrier layer 400. The absorbent layer 100 and top layer 300 are stitched to the fabric body 32. The attaching element 700 is an extension of the fabric body 32 and is attached to the top layer 300 via an adhesive film 500. The fabric body 32 and attaching element 700 include a liquid impermeable material or are treated with a hydrophobic material. The liquid impermeable fabric body 32 thus functions as the barrier layer 400 to prevent leakages from the bottom of the absorbent pad 20. The attaching element 700 and fabric body 32 cooperatively cover the visible stitch lines 900 and seal the periphery of the absorbent pad 20 from side leakages.

In one embodiment as shown in FIG. 9D, the absorbent pad 20 includes the absorbent layer 100 and top layer 300, but does not have the barrier layer 400. The absorbent layer 100 and top layer 300 are stitched to the fabric body 32. The sealing element 600 is attached to the top layer 300 and fabric body 32. Additionally, the attaching element 700 is attached to the sealing element 600 to cover the sealing element 600. The sealing element 600 and attaching element 700 cooperatively cover the visible stitch lines 900 and seal the periphery of the absorbent pad 20 from side leakages. Further, the fabric body 32 includes a liquid impermeable material or is treated with a hydrophobic material to function as the barrier layer 400 and prevent leakages from the bottom of the absorbent pad 20.

In one embodiment as shown in FIG. 9E, the absorbent pad 20 is stitched to the fabric body 32. The sealing element 600 is an extension of the barrier layer 400 and is attached to the top layer 300. Additionally, the attaching element 700 is an extension of the fabric body 32 and is attached to the sealing element 600 to cover the sealing element 600. The attaching element 700 covers the visible stitch lines 900 and the sealing element 600 seals the periphery of the absorbent pad 20 from side leakages.

In one embodiment as shown in FIG. 9F, the absorbent pad 20 is stitched to the fabric body 32, without stitching through the absorbent layer 100. The peripheries of the top layer 300 and absorbent layer 100 are attached by an adhesive film 500. The sealing element 600 is attached to the top layer 300 and barrier layer 400 to seal the periphery of the absorbent pad 20 from side leakages. This embodiment avoids the use of a separate piece of fabric material to cover the visible stitch lines 900.

In one embodiment as shown in FIG. 9G, the absorbent pad 20 is stitched to the fabric body 32. The sealing element 600 is an extension of the barrier layer 400 and is attached to the top layer 300 to seal the periphery of the absorbent pad 20 from side leakages. Additionally, the sealing element 600 is folded such that it covers the visible stitch lines 900.

In one embodiment as shown in FIG. 9H, the absorbent pad 20 is stitched to the fabric body 32. The sealing element 600 is an extension of the barrier layer 400 and is attached to the top layer 300. The attaching element 700 is an extension of the fabric body 32 and is attached to the sealing element 600 to cover the sealing element 600. The sealing element 600 and attaching element 700 cooperatively seal the periphery of the absorbent pad 20 from side leakages. Additionally, the sealing element 600 and attaching element 700 are folded such that they cover the visible stitch lines 900.

In one embodiment as shown in FIG. 91, the absorbent pad 20 is stitched to the fabric body 32. The sealing element 600 is an extension of the barrier layer 400 and is attached to the top layer 300. The attaching element 700 is an extension of the fabric body 32 and is attached to the sealing element 600 to cover the sealing element 600. The sealing element 600 and attaching element 700 cooperatively seal the periphery of the absorbent pad 20 from side leakages. Further, the absorbent pad 20 includes a second attaching element 720 attached, such as by stitching or bonding with an adhesive film 500, to the top layer 300 and the attaching element 700. Additionally, the sealing element 600, attaching element 700, and second attaching element 720 are folded such that they cover the visible stitch lines 900.

In one embodiment as shown in FIG. 9J, the absorbent pad 20 is attached to the fabric body 32. The attaching element 700 is attached to the peripheries of the top layer 300, absorbent layer 100, and barrier layer 400 via an adhesive film 500. Further, the attaching element 700 is stitched to the fabric body 32. Specifically, at least some of the stitch lines 900 pass through the attaching element 700, adhesive film 500, barrier layer 400, and fabric body 32, and at least some of the stitch lines 900 further pass through the absorbent layer 100.

In one embodiment as shown in FIG. 9K, the absorbent pad 20 is attached to the fabric body 32, similar to the embodiment of FIG. 9J. However, the attaching element 700 is longer than the adhesive film 500 such that part of the attaching element 700 is stitched directly to the fabric body 32. Specifically, at least some of the stitch lines 900 pass through the attaching element 700, barrier layer 400, and fabric body 32 without passing through the adhesive film 500.

In one embodiment as shown in FIG. 9L, the absorbent pad 20 is attached to the fabric body 32, similar to the embodiment of FIG. 9K. However, part of the attaching element 700 is stitched directly to the fabric body 32, such that at least some of the stitch lines 900 pass through the attaching element 700 and fabric body 32 without passing through the adhesive film 500 or barrier layer 400.

In some embodiments, the attaching element 700 may have a first portion and a second portion. The first portion may be bonded to the sealing element 600, and the second portion is detached from the first portion and is arranged to be attached to the fabric body 32.

In some embodiments, the absorbent pad 20 may include a foam material and may be moulded. The foam material may be provided in the final product of the absorbent pad 20 either in a moulded form or in an unmoulded form, depending on the desired final shape of the product. For example, the foam material may include thermosetting and/or thermoplastic polymers, such as a foam material that comprises from 50 to 100 wt % of polyurethane). Other suitable foam materials may include, but are not limited to, polyethylene foams, polypropylene foams, polyurethane foams, ethylene-vinyl acetate (EVA) foams, non-woven foams, and combinations thereof. Further examples of suitable foam materials include a mixture of the foams mentioned previously with a material like elastane. Alternatively or additionally, the absorbent pad 20 may be shaped using the suitable fabric materials and/or apparel constructions/techniques instead of or in addition to the foam material. For example, the fabric material may be a spacer fabric. For example, various techniques can be used on fabric material to form the desired shapes, such as moulding, sewing, stitching, and knitting. For example, darts may be sewn on the absorbent pad 20 to form the desired shape. For example, the fabric material, such terry fabric, may be knitted into the desired 3D shapes using suitable knitting methods such as flat knitting or circular knitting. Shaping of the absorbent pad 20 may offer a more organic fit that adheres to the body's natural curvature, allowing custom-made foam shapes that achieve better aesthetics and comfort to the eventual wearer. For example, the absorbent pad 20 may be shaped like a 3D pouch for male undergarments to increase comfort for male users.

The absorbent pad 20 may be used in various types of garments 30. The garments 30 disclosed herein may include any garment 30 that intended to be worn in direct contact with the user's skin, such as undergarments worn around the groin region. Specific examples of undergarments include a panty, boxers, hipsters, tights, leggings, a diaper, a nursing undergarment, and an incontinence undergarment. For example, an undergarment may include leggings, panties, boxers, hipsters, etc. In certain embodiments, the undergarments may be associated with sanitary care. In further embodiments, the undergarments may be associated with exercise clothing or for assisting users who may suffer from hyperhidrosis, who may need targeted absorption of bodily fluids during the course of the day and/or during exercise. Other examples of the garments 30 include absorbent underwear, leakproof swimwear, baby diapers, swim diapers, bras, shorts, swimwear, activewear, leggings, tights, and any garment 30 that can incorporate the absorbent pad 20.

As described above, the absorbent pad 20 includes absorbent layer 100 that is unitary due to the knitting of the various types of yarns 200, thereby minimizing the thickness of the absorbent layer 100 and the resultant absorbent pad 20. The thin absorbent pad 20 is more compact than existing pads and enhances comfort for the user. When attached to a garment 30, the absorbent pad 20 may be affixed so that they are fully attached to the fabric body 32 or may only be attached at certain points (e.g. at their lengthwise peripheries (in whole or in part) to provide a “floating” effect. This arrangement may make the absorbent pad 20 and garment 30 more stretchable, which improves comfort and freedom of movement for the user. Further, the absorbent pad 20 and/or garment 30 may include stretchable or elastic materials. For example, the waist opening and/or leg openings of the fabric body 32 may include a stretchable material. The stretchability of the absorbent pad 20 and garment 30 allows the garment 30 to be adjusted to the body shape of the user, thereby improving the fitting and reducing risk of leakages.

In the foregoing detailed description, embodiments of the present disclosure in relation to an absorbent pad and a garment comprising the absorbent pad are described with reference to the provided figures. The description of the various embodiments herein is not intended to call out or be limited only to specific or particular representations of the present disclosure, but merely to illustrate non-limiting examples of the present disclosure. The present disclosure serves to address at least one of the mentioned problems and issues associated with the prior art. Although only some embodiments of the present disclosure are disclosed herein, it will be apparent to a person having ordinary skill in the art in view of this disclosure that a variety of changes and/or modifications can be made to the disclosed embodiments without departing from the scope of the present disclosure. Therefore, the scope of the disclosure as well as the scope of the following claims is not limited to embodiments described herein.

1. An absorbent pad for use in a garment, the pad comprising:
a unitary absorbent layer having a first surface and an opposing second surface, the absorbent layer made of an absorbent fabric comprising:
a plurality of different yarns knitted together between the first and second surfaces, the plurality of different yarns comprising:
a plurality of first yarns running in a wale direction for wicking fluid;
a plurality of second yarns running in a course direction for distributing fluid along the fabric; and
a plurality of third yarns running in the course direction for absorbing fluid,
wherein the first yarns, second yarns, and third yarns are interconnected to each other such that:
fluid is communicable between the first yarns, second yarns, and third yarns;
the first surface comprises substantially of the first yarns; and
the second surface comprises substantially of the third yarns; and
a liquid impermeable barrier layer attached to the absorbent layer at the second surface for retaining the absorbed fluid within the absorbent layer,
wherein the first surface is arranged to face towards a skin of a user when in use.

2. (canceled)

3. The pad according to claim 1, wherein:
each of the first yarns adopts a looping structure; and
each of the second yarns is held in place by each loop of the first yarns that each of the second yarns contacts in the course direction.

4. The pad according to claim 1, wherein the third yarns are intermittently interlooped with the first yarns such that at least some of the third yarns extends outwardly from the second surface.

5. The pad according to claim 4, wherein the third yarns are intermittently interlooped with the first yarns from every second to every tenth loop of the first yarns in the course direction.

6. The pad according to claim 5, wherein:
the plurality of different yarns further comprises a plurality of fourth yarns running in the course direction for distributing fluid along the fabric; and
the first yarns, second yarns, third yarns, and fourth yarns are interconnected to each other, such that fluid is communicable between the first yarns, second yarns, third yarns, and fourth yarns.

7. The pad according to claim 6, wherein the second surface comprises substantially of the third yarns and fourth yarns.

8. The pad according to claim 7, wherein the fourth yarns are intermittently interlooped with the first yarns, such that the fourth yarns intermittently separate the third yarns.

9. (canceled)

10. (canceled)

11. (canceled)

12. The pad according to claim 6, wherein the first surface comprises substantially of the first yarns and fourth yarns.

13. The pad according to claim 12, wherein:
each of the first yarns adopts a looping structure; and
each of the fourth yarns is held in place by each loop of the first yarns that each of the fourth yarns contacts in the course direction.

14. The pad according to claim 6, comprising:
a set of absorbent islands; and
a set of wicking seas separating the absorbent islands from each other,
wherein each absorbent island comprises the first yarns, second yarns, and third yarns; and
wherein each wicking sea comprises the first yarns, second yarns, and fourth yarns.

15. (canceled)

16. The pad according to claim 6, comprising:
a set of wicking islands; and
a set of absorbent seas separating the wicking islands from each other,
wherein each wicking island comprises the first yarns, second yarns, and fourth yarns; and
wherein each absorbent sea comprises the first yarns, second yarns, and third yarns.

17. (canceled)

18. The pad according to claim 1, further comprising a top layer attached to the absorbent layer at the first surface.

19. The pad according to claim 18, wherein the top layer functions as a wicking layer to facilitate liquid transfer to the absorbent layer.

20. The pad according to claim 18, wherein the top layer comprises a hydrochromic material configured to change colour in response to contact with the liquid.

21. The pad according to claim 1, further comprising an attaching element for attaching the pad to a garment.

22. The pad according to claim 21, wherein the attaching element forms a liquid impermeable ridge on a periphery of the pad.

23. The pad according to claim 21, further comprising a plurality of fasteners attached to the attaching element, the fasteners configured for fastening the pad to the garment.

24. The pad according to claim 1, further comprising a foam material and/or a spacer fabric material for shaping the pad.

25. A garment comprising:
a fabric body;
the absorbent pad according to claim 1, the pad being attached to the fabric body; and
a liquid impermeable barrier layer being part of the fabric body and/or part of the absorbent pad.

26. The garment according to claim 25, wherein the pad and/or fabric body comprises a stretchable material.
